(19) United States
(12) Patent Application Publication
(10) Pub. No.: US 2021/0299246 A1
(43) Pub. Date: Sep. 30, 2021

(54) METHODS AND COMPOSITIONS FOR THERAPEUTIC TREATMENT OF VIRAL OR VIRALLY-INDUCED INFECTIONS AND CONDITIONS, AND ANTI-VIRAL COMPOSITIONS AND THEIR PRODUCTION

(71) Applicant: R. Brannon Claytor, Malvern, PA (US)

(72) Inventor: R. Brannon Claytor, Malvern, PA (US)

(21) Appl. No.: 17/214,845

(22) Filed: Mar. 27, 2021

Related U.S. Application Data
(60) Provisional application No. 63/000,884, filed on Mar. 27, 2020.

(51) Int. Cl. A61K 39/215; A61P 31/14; A61K 35/28; C07K 16/10; C12N 5/0775

(52) U.S. Cl. CPC A61K 39/215; A61P 31/14; A61K 2039/5156; C07K 16/10; C12N 5/0667; A61K 35/28

(57) ABSTRACT

Methods and compositions for treating a viral infection, where the cells from the host to be treated are extracted from the host and modified with viral DNA, and then replaced back to the host to generate an immune response. The composition may be produced using fat cells that are mechanically or chemically transformed to a nanofat, and co-incubation of the nanofat with the adenovirus. The double-stranded DNA of the adenovirus codes for a specific antibody to be produced, and the transfer of genetic material from the virus to the nanofat stem cells takes place outside of the host where the host immune system cannot destroy it. The co-incubated cells are then administered to the host by introduction into the nasal passages or directive cavity, or through injection or other vehicle. The compositions provide an effective rapid response to an invasive pathogen, such as an adenovirus or bacteria, through passive immunity.

### CROSS-REFERENCE TO RELATED APPLICATIONS

The benefits under 35 U.S.C. §§ 119(e) and 120 of the following are hereby claimed: U.S. Provisional Application Ser. No. 63/000,884, filed on Mar. 27, 2020, the complete contents of which are incorporated herein by reference.

### BACKGROUND OF THE INVENTION

### 1. Field of the Invention

The present invention relates to compositions and methods for inhibiting viral replication and treating viral infections, diseases and disorders in a mammalian host, including humans and animals, and more particularly, to methods and compositions that provide passive immunity to a mammalian host via adenoviral gene delivery of antibody to adipose tissue-derived stem cells.

### 2. Brief Description of the Related Art

Pathogens, such as, bacteria, viruses, and other microorganisms are encountered daily by individuals, and can cause disease or harmful conditions within the body. The body's immune system, which is made up of various organs, cells and proteins, protects the body from these harmful substances when encountered. In a healthy individual, with a normal functioning immune system, the responses are seamless and one may not even be aware that the immune system is fighting off invasive germs. However, when the body encounters certain Viruses that are particularly aggressive or when the body is in a weakened state or condition, it may become ill. An individual is likely to become ill when the individual encounters Viruses that the individual's body has not previously come in contact with. Some pathogens, such as varicella (i.e., chickenpox), and other presumed childhood diseases, are likely to only make an individual ill once, when the person first comes into contact with them. This is a result of the body developing an immunity to the pathogen.

The body's immune system fights disease-causing germs (pathogens) like bacteria, viruses, parasites or fungi, and functions to remove them from the body. The immune system also functions to recognize and neutralize harmful substances from the environment, as well as to fight disease-causing changes in the body, such as cancer cells.

When the body encounters a foreign or invasive substance, and does not recognize the substance as its own, the immune system is therefore activated. The immune system may be activated by a number of different things that an individual's body does not recognize. The substances that activate the immune system or trigger its response are known as antigens. Some examples of substances that are antigens include proteins on the surfaces of bacteria, fungi and viruses. The antigens attach to special receptors on the immune cells (immune system cells) and the body responds with a series of processes triggered by the presence of the antigen. The first time that the body encounters a particular pathogen, the usual process is for the body store information about the invading pathogen and how to fight it. This stored information enables the body to mount a response if it comes into contact with the pathogen again by enabling the recognition of the germ so it can seek to eradicate it faster.

The body has B-cell receptors and T-cell receptors that comprise part of the body's immune system. The T-cell antigen-recognition molecules are made of membrane-bound proteins. The T-cell antigen-recognition molecules only function to signal T cells for activation. T-cell receptors (TCRs) have a protein structure that includes a variable region (V region) and a constant region (C region). These receptors are related to immunoglobulins in protein structure and the genetic mechanism responsible for their variability. Unlike the B-cell receptor, the T-cell receptor does not recognize and bind antigen directly, but instead recognizes short peptide fragments of pathogen protein antigens that are bound to the major histocompatibility complex (MHC) molecules on the surfaces of other cells.

Immunoglobulins and T-cell receptors function as the antigen-recognition molecules of the body's adaptive immune response. The body's specific immune system is known as the adaptive immune system. The adaptive immune system is responsible for making antibodies that are used to fight particular pathogens that the body has previously come into contact with. Typically, the adaptive immune response is a learned or “acquired” immune response, and is specific for a particular pathogen, i.e., bacteria, virus or other microorganism. Because the adaptive immune system is constantly learning and adapting, the body can also fight bacteria or viruses that change over time.

Certain types of white blood cells also comprise the body's immune system. In addition, chemicals and proteins in the blood, such as antibodies, complement proteins, and interferon are part of the immune system and function to support an immune response. Lymphocytes are a type of white blood cell. There are B and T type lymphocytes and each has a specific role in the immune system. For example, T lymphocytes directly attack foreign substances in the body, while B lymphocytes work together to help the immune system cells. The B lymphocytes become cells that produce antibodies that can attach to a specific antigen and facilitate the process of destroying the antigen, while the T lymphocytes mount a direct attack on the antigen itself in order to control the body's immune response. In addition, the T lymphocytes control the immune response by the release of certain chemicals, referred to as cytokines. The lymphocytes learn to tell the difference between your own body tissues and substances that are not normally found in your body, and once formed, the B and T cells serve to provide a memory function for the body's immune system, so that the immune response to a previously experienced pathogen will be more rapid.

In the control of viruses, immunizations have been developed as a means for promoting the resistance to a known pathogen, such as a bacteria or virus, in mammals, including humans.

Immunization against viruses can be conveyed through active or passive immunization. Active immunity is when the immunity is developed by the host following exposure to the pathogen or the antigen. The antigen is seen as foreign and an immune response is mounted to defeat the virus. This can be through cell-mediated immunity where the cells that are infected express the viral antigen and are detected by the educated T-cells which then direct cytotoxic cells to kill the infected cell. This can take several days or even weeks to mature and become operational. This is why the flu vaccine is scheduled to be given before the flu season so that patients are able to develop the necessary antibodies and immune response in order to contain the virus.

Passive immunity is when the immunity is developed from antibodies from outside of the host. The most common type of passive immunity is when a pregnant mother passes on antibodies to the fetus either through the umbilical cord or through breast milk. There is no lag time with passive immunity as the antibodies are immediately available to bind with any virus and initiate the immune response which can envelope and destroy the virus by a process of phagocytosis (or cell envelopment and destruction of the virus). Immunoglobulins or Ig represent the antibodies available to protect the host from attack by antigens. The immune system has the ability to make antibodies to every conceivable antigen that nature may present to it. However, if the host has not seen the antigen expressed by the virus previously, it is unprotected. Therefore educating the host is optimal but the time it takes for the body to develop antibodies may not be sufficient to prevent the virus from taking hold of the host and leading to further secondary immune response which can result in pulmonary fibrosis and impair oxygen delivery across the alveolar oxygen to blood transfer and thus result in the patient literally suffocating in their own fluid.

Therefore, if the body could have a supply of antibodies specifically directed to an antigen, a passive immunity would be conveyed to the host and thus prevent any penetration of the virus past the mucosal defenses of the body. It is a major over simplification of the immunoglobulins, but the IgA exist mainly in the mucosal environment of the respiratory tract and the digestive tract, and the immunoglobulins IgM, IgG, IgE, and IgD are mainly in the circulatory system. They are produced by B cells and educated in the bone marrow based on antigens encountered. Since an endless number of combinations are possible, it is expected that periodically B cells will produce antibodies against the host. These are generally deleted once detected within the bone marrow. However, if they are not then a condition known as auto-immunity can develop. When passive immunity is delivered through the administration of serum or blood plasma from animals or other humans which have developed the antibodies to the antigen, it immediately binds to the target antigen and the body eliminates the antigen. In cases where the antibody alone is administered, it is a short time immunity which can last weeks or a few months until those antibodies are consumed.

In the field of plastic surgery, a process has been pioneered for the transfer of the host's own fat cells for volumization of areas of the body which have become volume depleted. These include but are not limited to fat injections to the face, buttock and breast. Fat can be removed from one part of the body and transferred to another part of the body. Since this is considered homologous use, the FDA permits such transfers. In an effort to minimize lumpy or irregular fat deposits, efforts have been made to mechanically break down connections between fibrous components of the fat cells. A by-product of this reduction of bulk of the fat grafts can result in the ultimate destruction of the larger fat cells. The fat is homogenized through repeated transfer between syringes via progressively smaller and smaller connectors. This results in the destruction of the fat cells. Finally, the cells are passed through a fine mesh screen. The remaining cells are ones which are smaller than 500 microns. This final viable product of mechanical cell separation is known as nanofat. The main component of nanofat is stromal vascular fraction (SVF) or Adipose tissue-derived stem cells (ADSC). These SVF and ADSC cells exist amongst fat cells and are densely adherent to the fat cells. They are believed to be progenitor or mesenchymal cells which have the ability to differentiate into over 200 different mature mammalian somatic cell types.

A need exists for a method and composition that can provide an immediate response to an infection, such as viruses or bacterial infections, and other organisms.

### SUMMARY OF THE INVENTION

Adenoviruses have a 50 year history of being able to utilized safely and effectively to deliver gene expression to cells. There are several other types of gene delivery techniques (retroviral, lentiviral, AAV, HSV) but the adenovirus uses double stranded DNA, has the highest titer and the lowest oncogenic potential and does not need the cells to be dividing for the gene to be incorporated, and thus may represent the highest utility for reliably generating gene expression to ADSC in the brief time period of co-incubation outside of the body.

By harvesting the ADSC/SVF and co-incubating the cells with the adenovirus and then re-introducing the ADCS/SVF, the host will not detect the homologous cell population as foreign, since the ADSC/SVF are autografts and once the DNA is incorporated, will start producing the proteins which code for the structural formation of the antibodies which are specific to bind with the targeted antigens.

Non gut mediated transfers of genes to Adipose tissue-derived stem cells (ADSC) permits transmission of genes and allows circumventing of the anti-adenoviral vector immunity which normally protects the body from virus infection.

One of the biggest shortcomings is the immunity against the adenovirus capsid. This is significantly avoided by introducing the gene into the cells outside of the body's immune system.

Once the adenovirus has incorporated the double stranded DNA into the ADSC by manner of co-incubation and mechanical stimulation or chemical separation, the cells may be injected into the host either as a spray into the mucosa of the digestive/respiratory tract or injected into the host.

The cells will then incorporate and begin producing the genes of the antibodies directed against the antigens. The viral attackers will be identified and neutralized or phagocytosed.

Methods and compositions are provided for treating a pathogenic infection in a mammal, including humans and animals, and for immunizing mammals, including humans and animals, against specific pathogens, such as viruses, bacteria, and other organisms. Preferred implementations target an adenovirus infection in a mammalian host. The method involves isolation of stromal vascular fraction (SVF) containing the Adipose tissue-derived stem cells (ADSC) from the host that is to receive the immunologic composition. According to preferred embodiments, the method and compositions involve isolation of the stromal vascular extract (SVF) to obtain the Adipose tissue-derived stem cells (ADSC) by mechanical disruption. Alternatively, the isolation of the stromal vascular extract (SVF) to obtain the Adipose tissue-derived stem cells (ADSC) may be carried out chemically with one or more chemical agents. In particular embodiments, the ADSC are obtained from fat cells and are co-incubated with the pathogen. Preferably, a process is used to obtain a nanofat from the host. The process may involve removal of fat (i.e., fat tissue) from an area of the body of the host. The removed fat is then mechanically broken down, and preferably to break the connections between fibrous components of the fat tissue and cells, or according to some alternate embodiments, the breakdown is accomplished chemically. The reduction of bulk of the fat extracted from the host results in the ultimate destruction of the larger fat cells. The fat that has been taken from the host is homogenized. According to preferred embodiments, the homogenization may be carried out through repeated transfer of the extracted fat between syringes via progressively smaller and smaller connectors. When the extracted fat has been subjected to the homogenization or repeated transfer through the syringes, then the fat cells are destroyed. The extraction sample that contains the cells is then passed through a fine mesh screen to separate the larger cells and components of the sample from the remaining cells, which are the nanofat. The smaller cells are smaller than the mesh size and therefore pass through the filter or screen. According to preferred embodiments, a 500 micron mesh screen is used, and the remaining cells are ones which are smaller than 500 microns.

According to preferred implementations, the stem cells comprise stem cells that are obtained from the mammalian host that is to be immunized using the method and compositions. Adenoviruses have double stranded DNA that codes for a specific antibody. Since the transfer of genetic material is occurring outside of the mammalian host's body, the adenovirus is less likely to be destroyed by the body's immune system. The progenitor nature of the ADSC allows for the development of this cell population to differentiate into B cells. Once the gene is incorporated, the cell can produce antibodies to confer passive immunity to the host against a specific antigen which resides on the surface of a virus, thus neutralizing the virus and protecting the host from infection.

The present method and compositions are useful for fighting off the infection of a virus, such as for example (COVID19), by transfecting an adenovirus which has the gene to make an antibody into the ADSC. These are then re-introduced into the mammalian host, which may be a human or an animal, and protect the host from the attacking virus (COVID-19) by making antibodies against the virus and thereby deliver passive immunity. Passive immunity therefore may be provided to people who are healthy but who are currently being prevented from going out and working. The method and compositions are designed to address and alleviate the problems and burdens behind the social distancing and “shelter in place” practices undertaken by individuals because people are trying not to “catch the coronavirus”. The methods and compositions deliver passive immunity to protect people who currently are not infected and allow them to safely return to work or carry out other functions, without worrying about getting “sick”. This passive immunity may also be helpful in combating the virus for people who are already infected by the virus as well.

### BRIEF DESCRIPTION OF THE DRAWING FIGURES

FIG. 1 is a flow diagram illustrating a schematic depiction of an exemplary implementation of a method for the treatment of a pathogen infection in a mammal according to the invention. Optional steps are depicted in broken lines, and each optional step may be implemented apart from any other optional step.

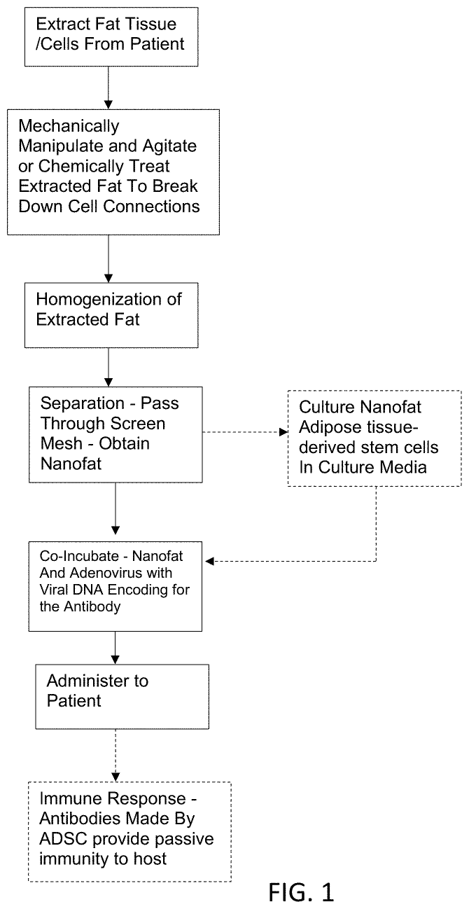

FIG. 2 is an exemplary illustration of a mechanism for producing antibodies from human stem cells.

### DETAILED DESCRIPTION OF THE INVENTION

Immunologic compositions are provided to target specific pathogens, such as bacterial, viruses and other organisms. According to a preferred implementations, the method for producing the composition and for treating a mammalian subject involves transforming cells obtained from the mammalian host subject. According to the method, cells are extracted from a mammalian host. The preferred implementations obtain the cells from the same host that is to be treated for the pathogenic infection. The host cells preferably comprise cells that contain the stem cells, in particular Adipose tissue-derived stem cells (ADSC).

Preferred implementations of the method are carried out using Adipose tissue-derived stem cells from the adipose tissue or fat tissue of the mammalian host. The cells obtained are preferably the Adipose tissue-derived stem cells (ADSC) found in the stromal vascular fraction (SVF) which is obtained from processing the fat sample extracted from the host. The fat sample take from the host may be a fat tissue sample. In the process, cells within fat tissue are separated from the actual gloppy fat tissue itself (the fat tissue being the fat that is initially extracted from the host). Those cells are then concentrated to make the SVF, from which transformation of the stem cells through incubation with the adenovirus encoded for the gene of the desired antibody which upon being returned to the host results in the immunologic composition.

According to preferred implementations, the fat cells are transformed through a procedure where the larger fat cells are chemically or mechanically broken down or destroyed by manipulation of the cells to break the connections between fibrous components of the fat cells. The reduction of bulk of the fat extracted from the host results in the ultimate destruction of the larger fat cells. The bulk reduced extracted fat sample from the host is processed to transform it further by homogenization to obtain the fraction of the cells desired for production of the immunologic composition that may be administered to the mammalian host. According to some preferred embodiments, homogenization of the extracted fat sample may be carried out through a repeated transfer of the extracted fat between syringes having progressively smaller and smaller connectors. The fat is passed through the series of syringes.

When the fat is destroyed through mechanical manipulation or a chemical treatment using these procedures, there is a fraction of very small cells in the sample. The smaller cell fraction is separated from the mechanically manipulated or chemically treated fat sample preferably by passing the mechanically manipulated or chemically treated fat sample through a screen that has a desired opening or mesh size to permit passage of the desired cell fraction that contains the desired Adipose tissue-derived stem cells, while leaving the other cells and cell components behind. According to preferred embodiments, a suitable screen mesh may be 500 micron. The fraction passing through the screen is collected, and contains the Adipose tissue-derived stem cells, and more particularly, the stromal vascular fraction (SVF) that includes the Adipose tissue-derived stem cells (ADSC). The Adipose tissue-derived stem cells obtained from the host fat and separated out preferably comprise progenitor or mesenchymal cells, which are capable of further development and replication. The procedures concentrate the progenitor cells in the sample.

According to preferred embodiments the virus is added directly to the nanofat (the fraction of the extracted fat tissue from the host that has been processed mechanically or chemically to break apart the fat cells and screened through a mesh screen, e.g., 500 micron screen). The co-incubation by the addition of the virus to the nanofat containing the stromal vascular fraction (SVF) that contains the Adipose tissue-derived stem cells (ADSC), transfers the viral DNA to the stem cells. The immunologic composition comprising the co-incubated host stem cells obtained from the host's processed pat tissue, is administered to the patient. The immunologic composition when placed in the patient's body, causes an immune response, and produces antigens targeting the virus.

The stem cell fraction obtained from the host, now containing the concentration of stromal vascular fraction (SVF) or Adipose tissue-derived stem cells (ADSC), is co-incubated with the pathogen. According to a preferred implementation, an adenovirus is the pathogen that is co-incubated with the stem cell fraction. Preferably, the co-incubated adenovirus is the target pathogen that the patient host (from whom the fat was extracted) is to be treated for, or immunized against, using the immunogenic composition. A suitable incubation procedure carried out to associate the adenovirus double stranded DNA and the stem cells of the stromal cell fraction containing the Adipose tissue-derived stem cells (ADSC) obtained from processing the extracted fat of the mammalian host.

Adenoviruses have double stranded DNA that codes for a specific antibody. Since the method involves the transfer of genetic material from the adenovirus to the extracted and processed host cells outside of the host body, the adenovirus is less likely to be destroyed by the body's immune system. The progenitor nature of the ADSC allows for the development of this cell population to differentiate into B cells. The adenovirus may be a particular adenovirus for which treatment of the infection in a mammalian host subject is desired. One example of a virus is the novel coronavirus, severe acute respiratory syndrome coronavirus 2 (SARS-CoV-2), which causes the infection coronavirus disease (COVID-19).

The co-incubation is carried out, and the immunogenic composition produced.

According to preferred embodiments, the immunogenic composition includes transformed cells of the host. The transformed host cells are capable of producing antibodies to the specific pathogenic antigen that was co-incubated with the Adipose tissue-derived stem cells. According to a preferred example, the immunologic composition produces antibodies that function against the adenovirus using the host patient body's immune system, when the immunologic composition is administered to the patient host.

The co-incubation of the virus and Adipose tissue-derived stem cells isolated from the fat (nanofat) promotes the development of the antibodies needed to attack the viral infection. The co-incubation is carried out on the extracted and processed host cells, without interference from the host's other cells and immune system response.

According to preferred embodiments, once the co-incubation has been carried out, the composition may be used as an immunologic composition by administering it to the host human or animal. According to preferred embodiments, the procedure may be carried out in a single host visit, to provide immediate passive immunity to the person or animal being treated.

The cell culture of the nanofat extracted cells may be co-incubated using a suitable procedure. For example, the co-incubation may be carried out by transfection or transduction.

According to some alternate embodiments, the nanofat extracted cells may be cultured for a period of time, e.g., a few to several days, prior to introduction of the adenovirus (viral DNA), or in the case of alternate embodiments, another pathogen.

According to some alternate embodiments, the nanofat cells that contain the SVF fraction (containing the Adipose tissue-derived stem cells (ADSC)) are cultured in a suitable culture media. The extracted nanofat cell fraction may be washed with a suitable washing agent, such as for example saline, e.g., a phosphate buffered saline, (e.g., Dulbecco's Phosphate Buffered Saline (DPBS), which may be commercially obtained (Invitrogen, Dun Laoghaire, Ireland]). The cell fraction may be centrifuged, and the centrifuged cells transferred to a container, such as a flask, along with a suitable culture medium that is designed to sustain the cells. For example, a suitable medium may comprise a human or animal sera, and a suitable cell growth supplement also may be added. Preferably, a human derived serum is utilized, as it may be advantageous over animal sera, and in addition, countries may restrict the use of animal serum during the production or treatment process. According to some preferred embodiments, human serum from type AB donors which is lacking in antibodies towards the A and B blood-type antigens, may be more preferred as immunoreactivity is minimized against the human cells in culture. In addition, male donor serum may be preferred over female in order to lower risks of major histocompatibility class (MHC) antibodies being present. (Women having a past pregnancy may have developed these antibodies to MHC antigens carried on the father's cells or the developing fetus.). Some examples of cell culture growth supplements include GroPro™ Cell Culture Growth Supplement (ZenBio, Inc.) and Human Platelet Lysate (HPL), which are non-animal derived cell culture growth supplements obtained from human platelets. HPL contains abundant growth factors and cytokines necessary for cell growth and proliferation. According to some embodiments, platelet lysate may be used as a replacement for traditional Fetal Bovine (FBS) supplemented cell culture medium. The aforementioned cell culture media are provided as examples, and other suitable culture media may be used. In some alternate applications, where a culture is carried out, the cells may be animal derived cells and may be fortified with growth factors or other supplements suitable for the animal host cells (e.g., when treating an animal).

During the cell culture period, the culture media may be changed and/or augmented as needed.

According to some alternate embodiments, the adenovirus is co-incubated to the cells of the cell culture. The cell culture preferably is compatible with the viral material antigen, such as the adenovirus, to permit the stromal cells to receive the viral DNA. The progenitor nature of the ADSC allows for the development of the cultured cell population comprising the extracted nanofat from the host to differentiate into B cells. During the co-incubation period, the gene is introduced into the cell population and incorporated into the cells. Once the gene is incorporated, the cell can produce antibodies that confer passive immunity to the host against a specific antigen which resides on the surface of a virus. The transfected nanofat cells of the host stromal vascular fraction (SVF) comprise an immunologic composition is then transferred to the host, which may be done by administering the composition (comprising the co-incubated cells) to the host via injection, spraying or other delivery means. According to some preferred implementations, the immunologic composition is administered to the host at preferred locations which include as a spray into the mucosa of the digestive and/or respiratory tract, or an injection into a site on the body of the host. The presence of the administered immunologic composition in the host neutralizes the virus and protects the host from infection.

Although the preferred methods are used without the need for culturing the cells and/or the use of a cell culture, according to some alternate embodiments a culture may be generated. In these embodiments, the stem cell fraction or stromal vascular fraction according to some embodiments may be cultured in a suitable culture media, which may also include nutrients and/or growth factors to permit the cells to stabilize and strengthen prior to co-incubation. A human derived culture media (versus animal derived media) is utilized according to preferred embodiments. According to some alternate implementations, the viral DNA may be introduced into a eukaryotic stromal vascular fraction (SVF) that contains the Adipose tissue-derived stem cells (ADSC) (the nanofat extracted cells) through chemical and physical methods in a physician's office or in a laboratory. According to some alternate embodiments, one or more additional agents may be used to promote the co-incubation, e.g., via transfection. For example, chemicals like calcium phosphate and diethylaminoehtyl (DEAE)-dextra may be used. Other agents which are commercially available and sold as transfection reagents may be used. The agents neutralize or may even impart an overall positive charge on DNA molecules so that the viral DNA molecule can more easily cross the negatively charged cell membrane of the stem cells. Alternatively, other methods such as physical methods, e.g., electroporation or microinjection which pokes holes in the cell membrane so DNA can be introduced directly into the cell may be used according to some embodiments. Microinjection requires the use of a fine needle to deliver nucleic acids to individual cells. Electroporation on the other hand uses electrical pulses to create transient pores in the cell membrane that genetic material can pass through.

According to some embodiments, a co-culture of the nanofat fraction and the virus may be generated by placing the nanofat fraction containing the patient's stromal cells and virus in the same tissue culture vessel (e.g., a plate, tube, or flask) without any barrier to impede contact between either the patient stromal cells and the virus or the medium in which they are cultured. In another embodiment, the nanofat cells and virus may be placed in the same tissue culture vessel but with a barrier that impedes contact between the them but does not impede transfer of the medium and any compounds smaller than the pore size of the barrier in which they are grown. Thus, the first and second cells can be exposed to the same tissue culture medium and/or any agents secreted by the nanofat cells or the virus (or the component agent that may contain the virus).

### Proposed Example 1

A mammalian patient having symptoms of, or who is confirmed to have a virus is treated for the infection. Cells comprising fat cells are extracted from the patient, by extracting a sample of fat tissue from the patient. In this example, areas proposed from which the fat cells are extracted include a suitable location, such as the buttock, cheek or breast of the patient. The tissue and cells therein are manipulated mechanically or chemically treated to disrupt and break apart the fibers of the fat cells. In the case of mechanical manipulation, according to a preferred implementation, the cells are mechanically manipulated by passing the extracted fat sample through a series of syringes having narrowing connectors, where the syringes are narrowing to impart forces on the extracted sample. The resultant substance containing the transformed fat cells is separated to obtain the desired nanofat fraction by passing the mechanically manipulated or chemically treated fat sample through a 500 micron mesh screen. The filtrate portion containing the Adipose tissue-derived stem cells in the nanofat is co-incubated with the virus to incorporate the viral DNA into the cells (Adipose tissue-derived stem cells (ADSC)) of the filtrate. The composition of the co-incubated fat cells and viral DNA are incubated at suitable temperature conditions for a suitable time. An immunologic composition is formed that includes Adipose tissue-derived stem cells that are capable of producing antibodies to the virus.

### Proposed Example 2

A patient was treated using the method set forth in Proposed Example 1, and the virus co-incubated is severe acute respiratory syndrome coronavirus 2 (SARS-CoV-2) that causes the coronavirus disease (COVID-19), and the immunologic composition treats the patient for coronavirus disease (COVID-19).

### Proposed Example 3

The patient treated in accordance with Proposed Examples 1, 2 or 3, was given a second administration of the immunologic composition after a period of time from the administration of the first treatment with the immunologic composition.

In accordance with the treatments provided by the methods and compositions disclosed herein, when the immunologic composition is administered to the patient, such as the mammalian host, the B lymphocytes (B cells) produce antibodies that can attach to the specific antigen of the pathogen being targeted and stimulate the process of destroying the antigen. When the antibodies attach to the specific antigen targeted by the method and composition it makes it easier for the host patient's immune cells to destroy the antigen.

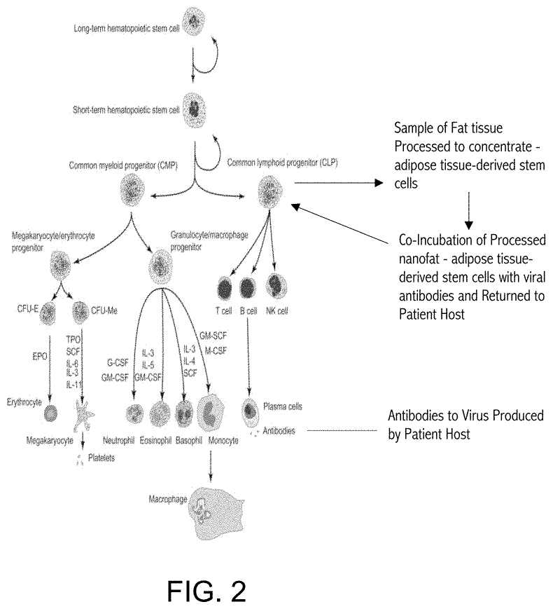

Referring to FIG. 2, a depiction of an exemplary diagram showing a stem cell, a progenitor cell, that may differentiate to form a B cell. The B cell is shown producing the antibodies that can confer immunity to the host against the specific antigen residing on the surface of the targeted virus. In FIG. 2, the gene or DNA of the virus may be incorporated so that the antibodies produced target the specific virus. FIG. 2 shows the formation of white blood cells. The differentiation of multipotent progenitors is represented, and there are two main lines. The lymphoid line is shown giving rise to T-lymphocytes and B-lymphocytes and natural killer cells. The viral co-incubation is represented and the antibodies produced by the B lymphocytes are also depicted.

### Proposed Example 4

Patients who do not have immunity to the virus and who have not been exposed could similarly be treated and be given Passive Immunity. There by allowing them to interact with others without the concern for “contracting” the virus as passive immunity exists.

In Table 1 below are common viral vectors that may be useful for co-incubation with the adipose tissue-derived stem cells according to the present method and compositions.

TABLE 1
Characteristics of the commonly-used viral vectors.

| | Viral system | | | | | |
|---|---|---|---|---|---|---|
| | Adenovirus | | | | | |
| | (Ad5) | AAV | Retrovirus | Lentivirus | HSV-1 | Baculovirus |
| Genome material | dsDNA | ssDNA | RNA | RNA | dsDNA | dsDNA |
| Genome size | 36 kb | 8.5 kb | 7-11 kb | 8 kb | 150 kb | 80-180 kb |
| Enveloped | No | No | Yes | Yes | Yes | Yes |
| Biosafety level | BSL-2 | BSL-1 | BSL-1/2 | BSL-2/3 | BSL-2 | BSL-1 |
| Insert size | 8-36 kb | 5 kb | 8 kb | 9 kb | 30-40 kb | No limit |
| | | | | | | known |
| Max titer | 1 × 10¹³ | 1 × 10¹¹ | 1 × 10⁹ | 1 × 10⁹ | 1 × 10⁹ | 2 × 10⁸ |
| (particles/ml) | | | | | | |
| Tropism | Broad, low | Broad, low | Broad | Broad | Neurons | Some |
| | for blood | for blood | (pan or | (pan or | | mammalian |
| | cells | cells | pseudo-typed) | pseudo-typed) | | cells |
| Infectivity | Dividing and | Dividing and | Dividing | Dividing and | Dividing and | Dividing and |
| | non-dividing | non-dividing | cells | non-dividing | non-dividing | non-dividing |
| | cells | cells | | cells | cells | cells |
| Transgene | Transient | Transient | Stable | Stable | Transient | Transient |
| expression | | or stable | | | | or stable |
| Vector genome | Episomal | Episomal | Integrated | Integrated | Episomal | Episomal or |
| form | | (>90%), site- | | | | integrated |
| | | specific | | | | |
| | | integration | | | | |
| | | (<10%) | | | | |
| Inflammatory | High (low | Low | Low | Low | High | High |
| potential | for “HC- | | | | | |
| | AdVs) | | | | | |
| Advantages | High titers; | Safe | Persistent | Persistent | Large | Large cargo |
| | extremely | transgene | gene transfer | gene transfer | packaging | sizes; high |
| | efficient | delivery; | in dividing | in most tissues | capacity; | level of gene |
| | transduction | non-inflammatory; | cells | | strong tropism | expression |
| | of most cell | non-pathogenic | | | for neuronal | |
| | types and | | | | cells | |
| | tissues | | | | | |
| Drawbacks | Capsid | Small | Only | Integration | Inflammatory; | Limited |
| | mediates a | packaging | transduces | might induce | no expression | mammalian |
| | potent | capacity; | dividing cells; | oncogenesis | during latent | host range |
| | inflammatory | requiring | integration | in some | infection; | |
| | response | helper AdV | might induce | applications | transient gene | |
| | (eliminated | for replication | oncogenesis | | expression in | |
| | in HC-AdVs) | and difficult | in some | | non-neuronal | |
| | | to produce | applications | | cells | |
| | | pure viral | | | | |
| | | stocks | | | | |

The preferred embodiments of the invention have been described in connection with fat cells from adipose tissue. However, according to some other embodiments, the host cells may comprise progenitor cells that are found in brain, bone marrow, blood vessels, skeletal muscle, skin, teeth, heart, gut, liver, and other (although not all) organs and tissues. However, the utilization of the fat cells is preferred.

Therapeutic immunologic compositions are produced as described herein, and preferably are generated from the host patient's own cells, and in particular the extracted fat sample. The processing and transformation of the fat sample to the nanofat containing the stromal vascular fraction (SVF) that contains the progenitor stem cells (adipose tissue-derived stem cells) provides a substrate fraction that is co-incubated with the virus. Once the co-incubation process has been carried out, the cells contain the DNA of the virus. This immunologic composition comprises host based cells that have been transformed through a mechanical manipulation process or chemical treatment to destroy the fat cells and dissociate fibrous bonds and that contain the DNA coding for the antibodies to the viral antigen. The immunologic compositions may be provided to target specific pathogens, to provide a rapid treatment for the patient host.

The methods and compositions may be applied to mammals that include animals. The method has utility not only for treatment of infections in a human host, but also may be useful to treat infections in farm animals, such as swine or bovine. The methods herein include methods for treating an infection in a mammalian host, as well as methods for producing an immunologic composition.

What is claimed is:

1. A method of delivering passive immunity against a pathogen to a mammalian host, comprising:
a) extracting fat tissue of the mammalian host to be immunized;
b) homogenizing the extracted fat tissue to extract the stem cell fraction (SVF) that contains the Adipose tissue-derived stem cells (ADSC);
c) separating the fat fractions to obtain the nanofat containing the Adipose tissue-derived stem cells (ADSC);
d) co-incubating the nanofat with the pathogen or pathogenic antigen to form an immunogenic composition; and
e) administering to said mammalian host the immunogenic composition.

2. The method of claim 1, wherein said nanofat comprises the stromal vascular fraction (SVF) of the fat tissue extracted from the host and contains Adipose tissue-derived stem cells (ADSC).

3. The method of claim 2, including culturing the extracted stem cells prior to co-incubation with said gene for the antibody.

4. The method of claim 2, wherein the adenovirus is co-incubated with the ADSC (extracted stem cells) directly.

5. The method of claim 1, wherein the gene coding for the antibody to said pathogen is an adenovirus and wherein said adenovirus codes for the gene of the antibody.

6. The method of claim 5, wherein administering the immunogenic composition to said host includes spraying the immunologic composition into the mucosa of the digestive and/or respiratory tract of the host, or injecting the immunologic composition into the host via injecting into the skin, subcutaneous tissue, muscle or into a blood vessel.

7. The method of claim 5, wherein co-incubation is carried out by transfection of said nanofat with said adenovirus.

8. The method of claim 7, wherein said adenovirus is an antibody specific to the corona virus (COVID-19).

9. The method of claim 8, wherein said coronavirus is severe acute respiratory syndrome coronavirus 2 (SARS-CoV-2) that causes the coronavirus disease (COVID-19).

10. The method of claim 8, including repeating step e) at a plurality of spaced apart time intervals.

11. An immunologic composition for treating a viral infection by a virus in a host comprising:
a modified host-generated stem cells transformed from nanofat having the viral DNA of the antibody directed against the viral infection to be treated, the modified host-generated stem cells being transformed to produce antibodies to the virus.

12. The composition of claim 11, wherein said modified host-generated stem cells comprise modified host-generated Adipose tissue-derived stem cells (ADSC) of the stromal vascular fraction (SVF) of fat cells from the host.

13. The immunologic composition of claim 11, wherein said viral DNA is an adenovirus DNA.

14. The immunologic composition of claim 11, wherein said adenovirus DNA is a gene that codes for the antibody against the adenovirus which is endangering the host.

15. The immunologic composition of claim 14, wherein said adenovirus DNA is a gene that codes for the antibody against the coronavirus which is endangering the host.

16. The immunologic composition of claim 15, wherein said coronavirus is severe acute respiratory syndrome coronavirus 2 (SARS-CoV-2) that causes the coronavirus disease (COVID-19).

17. A method of producing an immunologic composition for treating a viral infection in a mammalian host comprising:
a) extracting fat tissue of the mammalian host to be immunized;
b) homogenizing the extracted fat tissue to extract the stem cell fraction (SVF) that contains the Adipose tissue-derived stem cells (ADSC);
c) separating the extracted fat tissue in step b) to obtain the nanofat fraction containing the Adipose tissue-derived stem cells (ADSC) by passing the fat tissue through a 500 micro mesh screen;
d) co-incubating the nanofat fraction with the pathogen or pathogenic antigen to form an immunogenic composition that comprises antibodies against the virus;
e) wherein said nanofat comprises the stromal vascular fraction (SVF) of the fat tissue extracted from the host and contains Adipose tissue-derived stem cells (ADSC); and
f) wherein said viral infection to be treated is a virus that is attacking the host, or to which the host is susceptible.

18. The method of claim 17, wherein said viral infection to be treated is coronavirus (COVID19), and wherein said pathogen is coronavirus (COVID19) or pathogenic antigen is coronavirus (COVID19) antigen.

19. The method of claim 17, including an optional step of culturing the nanofat fraction passing through the 500 mesh screen in step c) that contains the Adipose tissue-derived stem cells (ADSC) in a cell-sustaining culture medium; and co-incubating the cultured nanofat fraction with the pathogen or pathogenic antigen to form an immunogenic composition that comprises antibodies to the virus.

20. The method of claim 1, wherein said mammalian host that receives the immunologic composition is asymptomatic or has no known prior exposure to the pathogen.

21. The method of claim 1, wherein said mammalian host that receives the immunologic composition is symptomatic or is confirmed to have an infection from the pathogen.

22. The method of claim 1, wherein homogenizing and/or separating the fat fractions to obtain the nanofat containing the Adipose tissue-derived stem cells (ADSC) comprises mechanical manipulation.

23. The method of claim 1, wherein homogenizing and/or separating the fat fractions to obtain the nanofat containing the Adipose tissue-derived stem cells (ADSC) comprises a chemical treatment.
